(19) United States
(12) Patent Application Publication
(10) Pub. No.: US 2007/0015267 A1
(43) Pub. Date: Jan. 18, 2007

(54) Method for producing composite objects using expanded graphite and vermiculite

(76) Inventors: Serge Da Silva, Perpignan (FR); Pierre Gros, Coloniers (FR); Cedric Leguen, Dremil Lafage (FR); Jacques Prosdocimi, Tournefeuille (FR); Sylvain Puybouffat, Balma (FR)

Correspondence Address: YOUNG & THOMPSON, 745 SOUTH 23RD STREET, 2ND FLOOR, ARLINGTON, VA, 22202, US

(21) Appl. No.: 10/574,803

(22) PCT Filed: Oct. 8, 2004
(86) PCT No.: PCT/FR04/02508

§ 371 Date: Apr. 5, 2006

(51) Int. Cl. B27N 3/00; C12N 11/04

(52) U.S. Cl. 435/182; 264/109

(57) ABSTRACT

A method for producing a composite object includes at least two distinct parts having different properties and/or functions, which includes forming at least one layer (53) having more than 70 wt. % of an expanded material selected from expanded graphites, forming at least one other layer (52) and more than 70 wt. % of another expanded material selected from expanded vermiculites, and then compressing the layers so formed together so as to consolidate them, each consolidated layer corresponding to one of the parts of the object. The method permits especially the production of composite objects such as an electrochemical cell, a casting mold or a heliothermal converter.

The invention relates to a method for producing composite objects comprising at least two distinct parts having different, even antagonistic, properties and/or functions.

These different properties and/or functions on the one hand require the use of different materials to produce said parts of the object and on the other hand lead to production methods which are often complex and long and which include especially steps of independently producing the two parts of the object and then assembling said parts.

Examples of known composite objects which can be mentioned include objects as diverse and varied as:
electrochemical cells which comprise, on the one hand, two electrodes having especially properties of electronic conduction and hydrophobicity and, on the other hand, an ion-exchange medium, such as water or a porous membrane, having properties of ionic (and not electronic) conduction and hydrophilicity, which medium separates the electrodes and includes an electrolytic solution. There is known especially the electrochemical cell described in WO 02/069415, the electrodes of which are each constituted by a flexible graphite sheet obtained from graphite which has been expanded and then laminated (uniaxial compression between pressure rollers according to a direction of compression denoted “c”), which sheet then undergoes a drawing operation which deforms its carbon planes (which are initially parallel) and creates transverse channels according to direction “c”, in order to remedy the disadvantages resulting from the anisotropy of Papyex® (electronic and thermal resistivity according to direction “c”). The face of the sheet that is to be in contact with the ion-exchange membrane then undergoes various treatments: catalytic treatment by deposition of a catalytic metal (noble metal such as platinum), hydrophobic treatment by means of a water-resistant additive, such as fluorinated polymers (and especially polytetrafluoro-ethylene, called Teflon®). In addition, the ion-exchange membrane is produced from sheets of glass, of porous polyolefins such as polyethylene, polypropylene, etc. The membrane and the electrodes then have to be assembled. The production of such a cell is obviously complex and long (large number of operations and treatments); it requires heavy and costly installations and employs a large number of materials (various polymers, noble metals, etc.);
casting molds, which are used to mold metal components from molten alloys and which comprise an internal refractory part which is to be in contact with the molten alloy. It can be advantageous to provide such molds with an external insulating part so that they can be handled during molding operations without risk of burning. There are known especially molds made of sand or another non-siliceous refractory material (zircon, chromite, olivine, bauxite). Such a mold is constructed in two parts, each corresponding substantially to one half of a model that is to be reproduced, by compression of sand in a frame. The sand is thus squeezed between the frame and the model, and then the model is removed. Cohesion of the sand is ensured by a binder, which is selected especially from wet clay, silica gels, synthetic resins, cements, etc., or by ceramics-type bonds which are created at high temperature. The frame is then lined with an external insulating case. In addition to the complexity of such a process, it should be pointed out that the handling of the sand is restrictive and dangerous (silicate powders, which are volatile, require the wearing of a mask) and that sand, which is certainly refractory, has some rather disadvantageous properties: its grained structure results in molds having a coarse surface quality which it is advisable to improve by additional finishing operations (for example polishing); its thermal properties do not allow the rate of cooling of the molten alloy to be controlled, and they can make the operations of pouring the alloy tricky and in some cases impair the mechanical characteristics of the resulting molded component; a mold made of sand can be used only once, and it is difficult, or even impossible, to recycle the sand owing to the presence of the binders;
heliothermal converters, the purpose of which is to convert solar radiation into thermal energy and which usually comprise:
an absorption plate which is capable of absorbing thermal solar radiation and conducting heat and which is generally made of copper, the face of which that is exposed to solar radiation, called the absorption face, is treated by anodization with black chromium (which is very toxic) so that it exhibits an improved absorption coefficient and selectivity,
a heat exchanger which is arranged beneath the plate and is formed by a tubular circuit transporting a liquid coolant. The tube(s) of the circuit are welded to the face of the plate opposite the absorption face, called the transfer face. The actual heat exchange surface is limited to the contact zone between the tube(s) and the transfer face, which zone is lineic for a tube of circular cross-section,
a case in which the plate and the exchanger are arranged and one face of which, oriented towards the sun, is made to be transparent to solar radiation and is preferably suitable for creating a greenhouse effect inside the case, while the other faces of the case are lined with an insulating complex formed by a layer of polyurethane foam inserted between two reflective aluminium films, in order to avoid any loss of heat through these faces. It is clear, given the complexity of such an object, that its production method is long and costly, because it comprises steps, uses heavy installations and employs expensive materials: production of the plate by molding, laminating, etc., anodizing treatment of the plate, production of the tubular circuit by extrusion, bending, etc., various anti-corrosion treatments for the plate and the circuit, production of the case, production of the reflective films, lining of the case by injection of polyurethane foam and provision of the reflective films, etc., assembly of all of the elements of the converter with, especially, welding of the exchanger circuit to the plate, etc.

The invention aims to propose a method of producing such composite objects which is simple, rapid and inexpensive.

In a preferred version, the invention aims especially to propose a method which allows two distinct parts of the object having different, even antagonistic, properties and/or functions to be formed and assembled in a single operation.

Another object of the invention is to provide composite objects the known properties and/or functions of which are improved or which offer new advantages.

The invention relates to a method for producing a composite object comprising at least two distinct parts having different properties and/or functions, which comprises:
forming at least one layer comprising more than 70 wt. % of an expanded material selected from expanded graphites,
forming at least one other layer comprising more than 70 wt. % of another expanded material selected from expanded vermiculites,
and then compressing the layers so formed together so as to consolidate them, each consolidated layer corresponding to one of the parts of the object.

It should be pointed out that the layers can be formed simultaneously or in succession in any order depending on the object to be produced. In the following, the term “layer of graphite” denotes a layer comprising more than 70 wt. % of an expanded material selected from expanded graphites; likewise, the term “layer of vermiculite” denotes a layer comprising more than 70 wt. % of an expanded material selected from expanded vermiculites. The state, consolidated or not consolidated, of the layer in question is indicated if necessary.

The inventive step governing the idea of the invention accordingly consisted, on the one hand, in choosing to use at least two expanded materials to produce distinct parts of an object having different functions and/or properties and, on the other hand, in imagining that it was possible to compress distinct layers of such materials together and thus obtain consolidation of each layer, despite the structural differences (in terms of crystalline arrangement, granulometry, mode of consolidation, etc.), mechanical differences (resistance to compression, viscosity, etc.), etc. between said materials. Contrary to all logic, the inventors have found that it was even possible to control the respective densities of the two layers, especially by selecting the granulometric profile of the expanded vermiculite used. It is thus possible to obtain a consolidated layer of vermiculite while the layer of graphite—which consolidates first and, before consolidation, absorbs the majority of the applied compression stress—has only a low density close to its minimum consolidation density (of the order of 30 kg/m³).

The inventors have also found, surprisingly, that it is possible by compressing two adjacent layers, that is to say two layers having faces in contact, to obtain consolidated layers which are joined together, including in the case of compression according to a single direction orthogonal with respect to said layers, that is to say orthogonal with respect to their interface. This result appears surprising considering that the graphite consolidates first and in an ordered lamellar structure, the parallel lamellae of which are able to slide relative to one another and confer a lubricating nature on the recompressed graphite, while consolidation of the vermiculite does not occur until after consolidation of the graphite and yields a non-ordered structure. It would therefore have been expected that the vermiculite, which additionally has a granulometry greater than that of graphite, would be unable to attach itself to the smooth and sliding surface of the consolidated layer of graphite. Joining takes place, however, and a slight intergrowth of the graphite planes and the vermiculite grains at the interface of the consolidated layers is noted a posteriori.

The method according to the invention allows two distinct parts of an object having different, even antagonistic, properties and/or functions to be produced by operations which are simple and low in number (formation and then simultaneous compression of layers). In particular, it allows the actions of forming and assembling said parts to be combined in a single operation. The method according to the invention is particularly simple and rapid; it requires little labour.

In addition to these advantages, it should be pointed out that the recompressed graphite and vermiculite are readily recyclable (they simply have to be exfoliated again by means of an intercalation solution).

Advantageously and in accordance with the invention, at least two of the layers are formed to be adjacent. By way of variation, the layers are formed in such a manner that they have no contact surface. That is the case if separating means (for example a separating sheet of a suitable material) are inserted between said layers or if the object to be produced comprises, in addition to its two distinct parts initially defined, a third part which is inserted between the first two parts and which is placed between the two layers before they are compressed.

In accordance with the invention, the layers formed are compressed together according to several directions, especially according to three orthogonal directions. By way of variation, the layers formed are compressed according to a single direction, especially according to a direction substantially orthogonal with respect to a front plane of one layer or with respect to an interfacial plane between said layers. The choice between these two methods depends substantially on the desired properties of the layer of graphite: uniaxial compression (compression according to a single direction) of the layers yields a consolidated layer of graphite that is highly anisotropic (the properties obtained according to compression direction “c” differ from those obtained according to any direction “a” orthogonal with respect to direction “c”), while compression according to all directions (the result obtained, for example, by compressing according to three orthogonal directions) yields a consolidated layer of graphite that is weakly anisotropic. By varying the compression stresses applied to the layers according to each direction, it is possible to adjust and control the properties of the consolidated layer of graphite.

In a version of the invention, the layers formed are subjected to a single compression operation according to each direction. In other words, the layers are compressed once according to each direction.

Advantageously and in accordance with the invention, the layers formed are subjected to a single compression operation, whether they are compressed according to a single direction or according to several directions simultaneously. In this case, the forming and assembly according to the invention of the two distinct parts of the object are carried out in a single operation.

By way of variation, the layers formed are subjected to a plurality of distinct compression operations according to at least one direction. In particular, there is carried out, according to that direction, a first compression operation suitable for consolidating the layers formed in order to allow them to be handled and, subsequently, a second compression operation suitable for conferring a desired density (greater than the minimum consolidation density) on one of said layers.

Advantageously and in accordance with the invention, during the compression of the layers formed there are impressed into at least one face, called an outer face, of at least one layer of graphite open recessed forms, called capture forms, which are suitable for trapping infra-red waves. The capture forms have especially at least one front dimension (opening) of from 1 μm to 1 cm and a depth of from 1 μm to 5 cm. The expression “outer face” is understood as meaning a face of the layer of graphite that is visible from the outside of the object, so that it can be exposed to a source of infra-red radiation.

By virtue of the optical selectivity and good thermal diffusivity of the recompressed expanded graphite, it is possible to regulate the temperature of the consolidated layer of graphite by exposing at least one of its outer faces to infra-red radiation. The presence of the capture forms improves the supply of calories by such radiant heating: an incident wave penetrating into a capture form undergoes multiple reflections at the opposing faces of the capture form; almost all the energy of the wave is finally absorbed by the graphite in such a capture form (the proportion of the incident flux which is reflected towards the outside of the form and which therefore may be lost is very small).

In addition, by increasing the surface area of the outer face, the presence of the capture forms also contributes towards facilitating not only the supply of calories but also the dissipation of calories in the case of cooling of the layer of graphite. Finally, the capture forms further reduce the thermal inertia of the layer of graphite, which is already low owing to the intrinsic properties of the recompressed expanded graphite.

The impressed capture forms can be linear impressions, such as slits, slots, grooves, etc., straight or curved impressions, impressions having a circular, square, triangular, etc. cross-section, or point-like impressions of pyramidal, conical, hemispherical, cylindrical (square or circular cross-section), etc. shape. The geometry of the impressed forms is chosen according to the wavelengths to be absorbed and the desired thermal response of the consolidated layer of graphite.

During compression of the layers formed, it is also possible to impress, into at least one outer face of a layer (of graphite or, preferably, of vermiculite), capture forms which are suitable for trapping sound waves or electromagnetic waves, according to the intended use of the object.

In all cases, the invention allows an object (or part of an object) to be provided with wave traps in a manner that is simple, rapid, economic, etc., without these additional functions of the object requiring the addition of supplementary members or means or the execution of an additional step in the production method. The wave traps are produced within the layers of graphite or vermiculite at the same time as these layers are consolidated.

Advantageously and in accordance with the invention, there is used as expanded graphite an expanded, optionally ground, natural graphite (but preferably a graphite as obtained after exfoliation).

It should be pointed out that, in accordance with the invention, in addition to comprising the expanded materials defined above, the layers formed can comprise additives selected from expanded or non-expanded materials, depending on the properties and functions of the object parts that are to be produced. In particular, the layer of vermiculite can comprise less than 30 wt. % additives selected from perlite, expanded materials obtained from oxides such as SiO₂ or Al₂O₃, kandites, illites, smectites, kaolinites.

The invention relates in particular to a method for producing an electrochemical cell. For this application, a layer of expanded vermiculite is formed between two layers of expanded graphite, and then the layers so formed are compressed together. Each consolidated layer of graphite forms an electrode, while the consolidated layer of vermiculite forms an ion-exchange membrane between said electrodes. Advantageously and in accordance with the invention, each layer of expanded graphite formed comprises less than 20 wt. % of a powder of a catalytic material (for example metal or metal oxide) in order to catalyse and/or promote electrochemical reactions.

The electrochemical cell produced in accordance with the invention is a so-called dry cell: the electrolytic solution—or the solvent when the crystalline reagents are disposed beforehand in the layer of expanded vermiculite—is added as required at the time the cell is used. Impregnation of the membrane can readily be carried out by any suitable means, for example with the aid of a pipette.

It should be pointed out that such a dry cell can be used as an electrochemical reactor, as a fuel cell, but also as a bioelectrochemical cell such as a cell for measuring the level of glucose in the blood. In known cells for measuring the glucose level, the deposition of a drop of blood on a removable tab provided for that purpose leads to the activation of an enzyme present on an electrode of the cell and the subsequent generation of an electric current. The activation of the enzyme and the intensity of the current produced are directly proportional to the level of glucose present in the blood. In earlier known cells, the enzymes, which are adsorbed in lyophilized form at the surface of an electrode, finish up by becoming detached. Advantageously and in accordance with the invention, the layer of vermiculite formed comprises lyophilized enzymes. In other words, the enzymes are mixed directly with the expanded vermiculite before the layers are formed and compressed, so that they are fixed inside the consolidated layer of vermiculite (membrane); the useful life of the cell is prolonged considerably.

Advantageously and in accordance with the invention, the layers formed are compressed together according to three orthogonal directions in order to reduce the anisotropy of the layers of graphite and thus obtain electrodes having good electronic conductivity in all directions, including in a direction orthogonal with respect to the interfacial plane between the electrode and the membrane.

Advantageously and in accordance with the invention, the layers formed are compressed in such a manner that the two consolidated layers of graphite have a density of from 30 to 60 kg/m³. In other words, the layers are lightly compressed in order to permit the provision of porous graphite electrodes. The electrochemical reaction at each electrode is accordingly carried out not only at the interface between the graphite electrode and the vermiculite membrane, but also inside the electrode itself. The specific surface at which the electrochemical reactions take place, and consequently the efficiency of the electrochemical cell, are increased considerably. The invention therefore also allows the properties of the object parts that are produced to be improved.

Advantageously and in accordance with the invention, by way of variation or in combination, for at least one of the layers of graphite, microgrooves are formed on one face of said layer, called an inner face, that is oriented towards the layer of vermiculite. To this end, threads which are destructible (destructible by heating or by chemical reaction, etc.) or removable are placed between the layer of expanded graphite and the layer of expanded vermiculite during their formation, which threads are destroyed or removed once the layers have been consolidated. The microgrooves permit not only an increase in the specific reaction surface on the electrode but also the formation, at the interface between the electrode and the ion-exchange membrane, of microchannels which are used to create a circulation of electrolytic reagents along the electrode and to permit continuous operation of the electrochemical cell.

Advantageously and in accordance with the invention, heating/cooling members, for example hydraulic or electric heating/cooling members, are incorporated into at least one of the layers of expanded graphite during its formation, in order to effect thermal regulation of the reagents.

Heating can also be effected without contact by means of at least one infra-red generator located outside and at a distance from the electrochemical cell, by virtue of the optical selectivity of the recompressed expanded graphite. In that case, capture forms are preferably impressed into at least one outer face of at least one of the layers of graphite in order to increase the absorption of infra-red radiation. The impressed outer face is typically the front outer face of the electrode, opposite the inner face oriented towards the membrane. In this application, the impressed capture forms preferably have at least one front dimension (opening) of from 1 μm to 5 mm and a depth of from 1 μm to 1 mm.

The invention accordingly provides the possibility of regulating the temperature of the reagents in the cell and therefore controlling the kinetics of the reactions that are being carried out and improving the efficiency considerably by controlling certain limiting factors: dissipation of the calories produced in the case of an exothermic reaction, supply of calories in the case of an endothermic reaction.

The invention relates also to a method for producing a mold, especially a casting mold. For this application, a model (the form which it is desired subsequently to reproduce using the mold) is covered with a layer of expanded graphite, then a layer of expanded vermiculite that covers at least part of the layer of graphite is formed, and then the layers so formed are compressed together. The layer of graphite, which is refractory, corresponds to the part of the mold that is to receive the molten metal alloy; the layer of vermiculite forms insulating protection on at least part of the mold and allows the mold to be handled without risk of burning. It should be pointed out that the invention is applicable to the production of any type of mold (it is applicable whatever material is to be received in the mold—thermosetting resin, etc.), but it is particularly advantageous when insulating protection of the mold is desirable.

In addition to its simplicity and rapidity of execution, the method according to the invention has a large number of advantages: the possibility of producing molds of complex shapes in a single operation; the dimensional accuracy and excellent surface quality of the resulting molds, allowing the conventional finishing operations (machining, polishing, etc.) to be omitted; the ease of removal from the mold owing to the lubricating nature of the recompressed expanded graphite; the long useful life of the resulting molds, which can be used very many times and retain especially a good surface quality despite the thermal and, optionally, chemical (corrosion, oxidation, etc.) attack to which they are subjected; the possibility of controlling the rate of cooling and solidification of the molten alloy, as will be explained hereinbelow.

Advantageously and in accordance with the invention, the layers formed are compressed together in such a manner that the consolidated layer of graphite has a density greater than 40 kg/m³ for a mold intended for low-temperature applications (materials to be molded of the plaster, plastics, elastomer type) and, preferably, a density greater than 100 kg/m³ for a mold intended for high-temperature applications, such as a casting mold (materials to be molded of the molten alloy type), a density greater than 40 kg/m³ ensuring, under any circumstances, total impermeability of the mold to the finest and most liquid materials to be molded, and a particularly fine surface quality. It should be pointed out that a density greater than 100 kg/m³ is preferred in the case of a mold for high-temperature applications because it confers better thermal diffusivity on the consolidated layer of graphite and therefore provides the possibility of effectively controlling the temperature of the mold and the rate of cooling of the material to be molded.

Advantageously and in accordance with the invention, heating/cooling members, such as a portion of an electric or hydraulic circuit, are placed in the layer of expanded graphite during its formation. The compression stresses applied in this case are chosen to be sufficiently low that they do not damage the heating/cooling members and, especially, to be suitable for conferring on the consolidated layer of graphite a density of less than 400 kg/m³. Because the expanded graphite is a good conductor of heat and has excellent thermal diffusivity, the heating/cooling means are used to regulate the temperature of the mold and therefore control the rate of cooling and solidification of the molten alloy.

By way of variation there is formed, directly in the mass of graphite, at least one channel which is suitable for receiving a heating/cooling fluid and which therefore constitutes (with the fluid and means for circulating and heating/cooling said fluid) means for heating/cooling the mold. To this end, at least one tube which is destructible (by melting or chemical reaction, etc.) or removable (the tube in this case is provided to be flexible in order to allow it to be removed whatever the shape of the channel that is formed) is placed in the layer of expanded graphite during its formation, said tube(s) being destroyed or removed once the layer of graphite has been consolidated. The compression stresses are chosen to be sufficiently high to obtain a graphite density that confers tightness and mechanical strength on each channel formed. Accordingly, the layers are preferably compressed in such a manner that the consolidated layer of graphite has a density greater than 150 kg/m³.

Heating of the mold, and, generally, regulation of the temperature thereof, can likewise be carried out without contact by means of infra-red generators located at a distance from the mold. In this embodiment, the layer of vermiculite is formed so as not to cover all of the faces of the layer of graphite and to leave uncovered at least one face thereof, called an outer face, which is visible when the mold is in use, in order to expose it to infra-red radiation. Advantageously and in accordance with the invention, capture forms are impressed into at least one outer face of the layer of graphite. In this application, the impressed capture forms preferably have at least one front dimension (opening) of from 1 mm to 2 cm and a depth of from 1 mm to 10 cm.

The invention relates also to a method for producing a heliothermal converter. In this application, a layer of expanded graphite is formed, in which there is provided at least one channel suitable for receiving a liquid coolant, a layer of vermiculite is formed, which layer covers at least part of the layer of graphite and leaves uncovered at least one face thereof, called an absorption face, and then the layers so formed are compressed together. In order to obtain the channel(s), at least one permanent tube, for example a metal tube and especially a tube made of copper, is placed in the layer of expanded graphite, the expression “permanent tube” being understood to mean a tube that is suitable for subsequently receiving a liquid coolant and that is left in the consolidated layer of graphite. By way of variation, at least one destructible or removable tube is placed in the layer of expanded graphite, said tube(s) being destroyed or removed once the layer of graphite has been consolidated.

The consolidated layer of graphite, at least one face of which is not covered by the vermiculite and is therefore able to absorb solar radiation, produces a particularly efficient heat exchanger. The recompressed expanded graphite has excellent thermal diffusivity (better than that of the metals that are currently used) and very low thermal inertia (improved response time). The consolidated layer of vermiculite forms an insulating plate or case.

The compression ratio of the layers is chosen in dependence on the desired thermal and mechanical properties of the layer of graphite. In the case where the channels for liquid coolant are formed directly in the mass of graphite (using destructible or removable tubes), the compression ratio is chosen to be sufficiently high to confer on the assembly the correct mechanical strength, which is compatible with the stresses to which it is subjected (pressure of the fluid in the channels, hydraulic connection, expansions and contractions due to large variations in temperature (summer-winter)). In particular, the layers are compressed in such a manner that the layer of graphite has a density greater than 150 kg/m³.

In the case where the channels for liquid coolant are constituted by inserted tubes (for example metal tubes), it is not necessary for the graphite to contribute to the mechanical rigidity of the assembly. It is then preferred to obtain a gain in weight and an improvement in the thermal properties of the exchanger and the energy yield of the converter: the compression ratio can be chosen to be relatively low. In particular, the layers are compressed in such a manner that the layer of graphite has a density less than 400 kg/m³.

It should be pointed out that, in both cases, the layers are preferably compressed once and according to a single direction, in order to simplify the production method.

In addition to its simplicity and rapidity of execution, the method according to the invention has the advantage of providing a heliothermal converter having improved efficiency, given the advantageous thermal properties of the recompressed expanded graphite and the structure of the heat exchanger itself. Whether the channels of the exchanger are formed in the mass of the graphite itself or are constituted by inserted tubes, the heat exchange surface corresponds to the totality of the cylindrical surface of the channels and not to a single line of contact between a cylindrical surface and a plate, as is the case in earlier converters. Moreover, the method according to the invention permits the omission of any toxic or dangerous electrochemical treatment in order to obtain selective or improved absorptivity (which treatment is at present carried out using chromium oxide, which is highly toxic and prohibited at term) and provides a converter that is more light-weight, simpler and more reliable. The risks of malfunctioning and the maintenance costs of the converter are reduced considerably.

The optical selectivity of the graphite absorber for infra-red also promotes the greenhouse effect necessary for heliothermal conversion. Advantageously and in accordance with the invention, during compression of the layers formed, capture forms suitable for trapping solar infra-red radiation are impressed into the absorption face of the layer of graphite. The impressed forms preferably have at least one front dimension (opening) of from 10 μm to 1 cm and a depth of from 1 mm to 1 cm.

The capture forms increase considerably not only the absorption surface of the graphite absorber but also the geometrical optical selectivity for wavelengths in the infra-red range.

They also allow the rigidity and the mechanical strength of the consolidated layer of graphite to be reinforced punctually, and consequently globally, by increasing the graphite density close to each capture form.

It is preferable to offset the capture forms with respect to the channels for liquid coolant, not only in order to limit the compression stresses on the channels (which stresses would be increased by the presence of the impression matrices of the capture forms facing the channels) but also in order to optimize the mechanical and thermal profile of the consolidated layer of graphite.

The invention extends to a composite object comprising at least two distinct parts having different properties and/or functions, wherein one of the parts comprises a consolidated layer comprising more than 70 wt. % of a recompressed expanded material selected from expanded graphites, and another of the parts comprises another consolidated layer comprising more than 70 wt. % of another recompressed expanded material selected from expanded vermiculites. Such an object can advantageously be obtained by a method in which the layers of graphite and vermiculite are compressed together and which preferably comprises only a single compression step (uniaxial or multiaxial).

The invention relates in particular to an electrochemical cell which comprises at least one consolidated layer of recompressed expanded vermiculite inserted between two consolidated layers of recompressed expanded graphite. The consolidated layers of graphite preferably have a density of from 30 to 60 kg/m³, so that they are porous. Advantageously and in accordance with the invention, at least one of the consolidated layers of graphite has microgrooves on one face, called an inner face, that is oriented towards the layer of vermiculite. Advantageously and in accordance with the invention, at least one of the consolidated layers of graphite incorporates heating/cooling members. By way of variation or optionally in combination, at least one of the consolidated layers of graphite has, on at least one outer face, impressed open recessed forms, called capture forms, which are suitable for trapping infra-red waves and which preferably have at least one front dimension of from 1 μm to 5 mm and a depth of from 1 μm to 1 mm. Advantageously and in accordance with the invention, the consolidated layer of vermiculite comprises lyophilized enzymes.

The invention relates also to a mold, especially a casting mold, which comprises at least one consolidated layer of recompressed expanded graphite delimiting a cavity corresponding to an object to be reproduced by molding, and a consolidated layer of recompressed expanded vermiculite covering at least part of said layer of graphite. The consolidated layer of graphite preferably has a density greater than 40 kg/m³, especially greater than 100 kg/m³ in the case of a casting mold. Advantageously and in accordance with the invention, the consolidated layer of graphite incorporates heating/cooling members, such as a portion of an electric or hydraulic circuit. By way of variation (or optionally in combination), the consolidated layer of graphite comprises at least one channel which has been formed directly in the mass of the graphite and is suitable for receiving a heating/cooling fluid. By way of variation or optionally in combination, the consolidated layer of graphite has at least one face, called an outer face, that is visible when the mold is in use; and at least one outer face of said layer comprises impressed open recessed forms, called capture forms, which are suitable for trapping infra-red waves and which preferably have at least one front dimension of from 1 mm to 2 cm and a depth of from 1 mm to 10 cm.

The invention relates also to a heliothermal converter which comprises at least one consolidated layer of recompressed expanded graphite comprising at least one channel suitable for receiving a liquid coolant, and a consolidated layer of recompressed expanded vermiculite covering the layer of graphite with the exception of at least one face thereof. Each channel extending in the consolidated layer of graphite has either been formed directly in the mass of graphite or is constituted by a tube, for example a tube made of copper, accommodated in the layer of graphite. The consolidated layer of graphite advantageously has, on its absorption face, impressed open recessed forms, called capture forms, which are suitable for trapping infra-red waves and which preferably have at least one front dimension of from 10 μm to 1 cm and a depth of from 1 mm to 1 cm.

The invention relates also to a method for producing a composite object and to such an object, which method and object, in combination, have all or some of the features mentioned hereinabove and hereinbelow.

Other aims, features and advantages of the invention will become apparent upon reading the following description, which refers to the accompanying drawings showing preferred embodiments of the invention which are given solely by way of non-limiting examples. In the drawings:

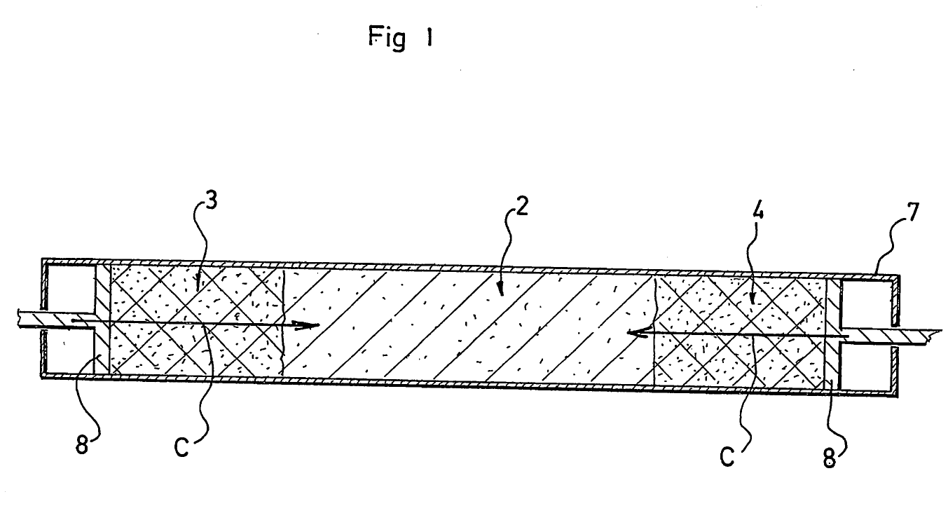

FIG. 1 is a cutaway view, in diagrammatic form, of a press used in accordance with the invention to produce an electrochemical cell,

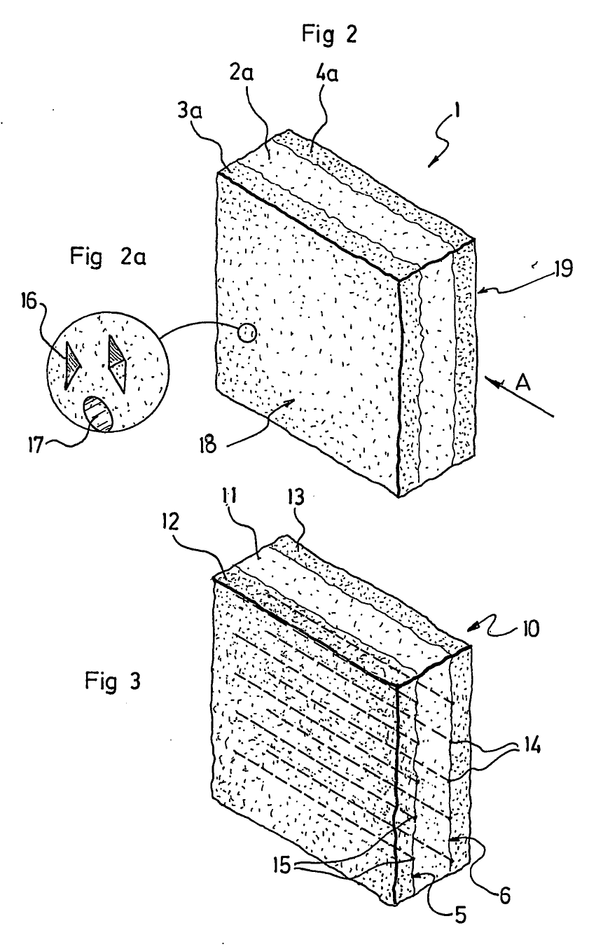

FIG. 2 is a perspective view of an electrochemical cell according to the invention,

FIG. 2a is a perspective view of a detail of an outer face of the electrochemical cell of FIG. 2,

FIG. 3 is a perspective view of another electrochemical cell according to the invention,

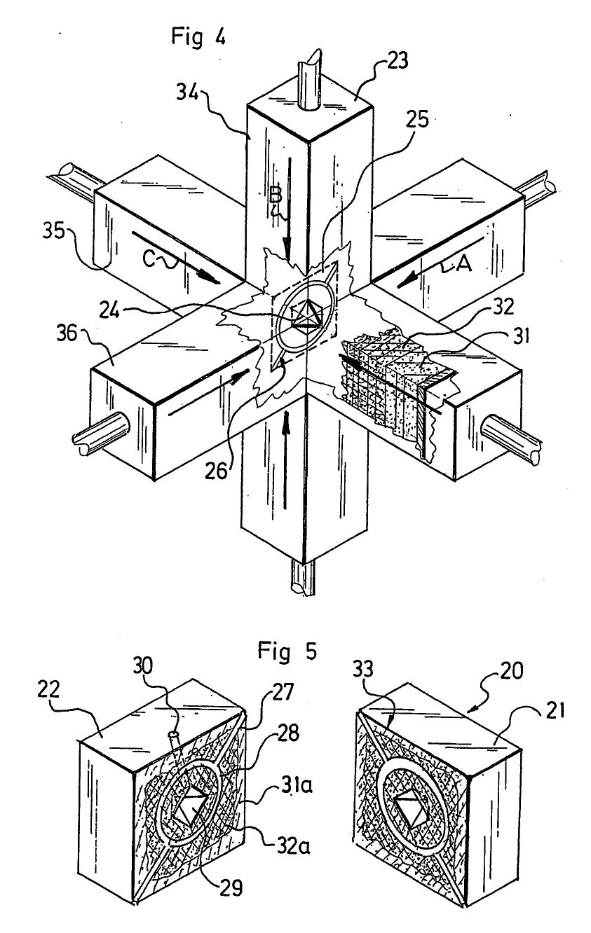

FIG. 4 is a perspective, partially cutaway view, in diagrammatic form, of a press used in accordance with the invention to produce a mold,

FIG. 5 is a perspective view of a mold in two parts according to the invention,

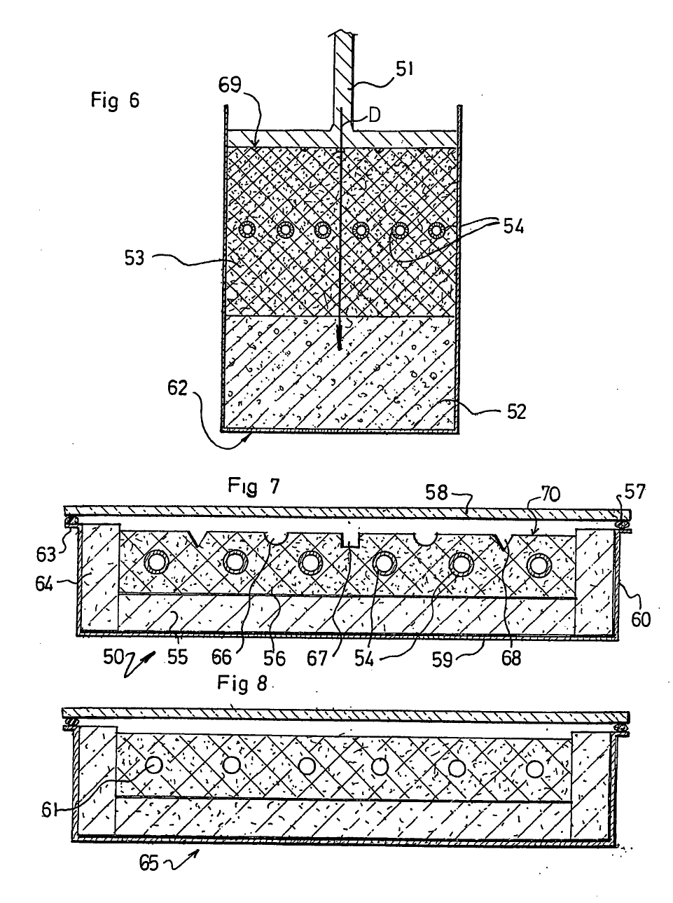

FIG. 6 is a cutaway view, in diagrammatic form, of a press used in accordance with the invention to produce a heliothermal converter,

FIG. 7 is a cutaway view of a heliothermal converter according to the invention,

FIG. 8 is a cutaway view of another heliothermal converter according to the invention.

As is shown in FIG. 1, the method for producing an electrochemical cell 1 according to the invention comprises forming, in succession, in a uniaxial press 7, for example, a layer 3 of expanded graphite, a layer 2 of expanded vermiculite and a layer 4 of expanded graphite. In the non-limiting example shown, the layers are superposed in the direction of compression of the press, denoted C. The material used to form each layer of graphite comprises more than 70 wt. % expanded natural graphite; it further comprises less than 20 wt. % of a powder of a catalytic metal (for example platinum) or of a catalytic metal oxide, distributed homogeneously in the expanded natural graphite. The material used to form the layer of vermiculite comprises only expanded vermiculite.

The above-mentioned three layers are then compressed together by actuation of at least one of the plates 8 of the press until the layers are consolidated. Preferably, the imposed compression ratio is low, that is to say just greater than both the minimum compression ratio necessary to consolidate a layer of graphite and the minimum compression ratio necessary to consolidate a layer of vermiculite. The layers are accordingly compressed in such a manner as to obtain two consolidated layers of graphite 3a and 4a (see FIG. 2) having a density of the order of from 35 to 40 kg/m³ in order to obtain porous consolidated layers of graphite and vermiculite.

The electrochemical cell 1 that is obtained is shown in FIG. 2. It comprises two electrodes 3a and 4a corresponding to the two consolidated layers of graphite, between which there extends an ion-exchange membrane 2a corresponding to the consolidated layer of vermiculite. The cell obtained is parallelepipedal if the press 7 has a square or rectangular cross-section. It has dimensions which can vary from 10 to 100 mm depending on its use. It should be pointed out that, above 100 mm, the weakly consolidated layers lack mechanical strength. It is then necessary to compress the layers in such a manner as to obtain densities greater than 60 kg/m³. The electrodes are less porous and the cell is less efficient. When a cell of large size is desired, it is therefore preferable to juxtapose a plurality of small (weakly compressed) cells, arranged in parallel or in series.

The outer face 18 of the electrode 3a and the outer face 19 of the electrode 4a have capture forms 16, 17 (see FIG. 2a) which have geometry and dimensions suitable for trapping infra-red waves. The capture forms are point-like cylindrical indentations whose cross-section is circular (such as 17), triangular-base-pyramidal (such as 16) or square or circular, etc., or marks of much more complex shape. The capture forms have, in the example shown, front dimensions of from 1 to 100 μm and a depth of from 1 to 100 μm. The capture forms are obtained by means of impression matrices which are carried by the plates of the press 7 (the matrices are not shown, given the scale) and have a plurality of point-like elements corresponding to said forms. The forms are impressed at the time of compression and consolidation of the layers 2, 3 and 4. In view of the properties of graphite, it is possible to obtain forms having microscopic dimensions with extreme dimensional accuracy and therefore to design very effective wave traps which are suitable for the source of radiation that is used. Such microscopic forms further increase considerably the exchange surface and therefore the efficiency of radiant heating or cooling.

The cell that is produced is a so-called dry cell, referring to the exchanger medium (the vermiculite membrane), which is dry and devoid of aqueous solution. The vermiculite membrane is impregnated with an electrolytic solution at the time the cell is used. By way of variation, the crystalline chemical reagents (in particular oxidation-reduction couple), preferably in the form of solid salts, are mixed with the expanded vermiculite (or with the composite material used to form the layer of expanded vermiculite) before the layers are formed, so that the reagents are present in the membrane as soon as it is produced. The membrane is impregnated with a solvent at the time the cell is used.

The electrochemical cell can be used as an electrolytic reactor, in which case each electrode 3a, 4a is connected to a terminal of an electric current generator by any suitable means (a conductive wire can be sealed directly into each layer of graphite during its consolidation). The voltage applied by the generator between the two electrodes imposes on each electrode a potential that is different from its equilibrium potential and leads to displacement of the oxidation-reduction reaction of the electrolytic solution in the direction of oxidation at the anode (electrode connected to the positive terminal of the generator, the imposed potential of which is greater than the equilibrium potential) and in the direction of reduction at the cathode (electrode connected to the negative terminal of the generator, the imposed potential of which is lower than the equilibrium potential).

The cell can likewise be used as a fuel cell. In this case, each electrode 3a, 4a is connected, by any suitable means, to a terminal of a current accumulator which is to store the current generated between the electrodes by the electrochemical reaction.

Because the graphite electrodes 3a and 4a are porous, the oxidation-reduction reaction at each of the electrodes takes place not only at the inner face of the electrode oriented towards the vermiculite membrane 2 but also, and primarily, in the mass of the electrode itself. The specific reaction surface is therefore reduced and increased considerably relative to a known electrode having the same outside dimensions. It should be pointed out that the catalytic metal is mixed directly with the expanded graphite before the corresponding layers are formed and is therefore present throughout the mass of the electrodes, at each potential reaction site. This method offers two major advantages: the efficiency of the cell is improved considerably (relative to known cells); the method is particularly simple and rapid, in contrast to the earlier methods in which a metal coating is produced on one face of the electrode by electrolytic deposition.

For continuous operation of the cell (reactor or cell), it is recommended to supply said cell with electrolytic solution. To this end, a circulation of electrolytic solution through the cell is created. It is possible especially to create a circulation of solution in each porous electrode, in particular according to a direction A, given that electrodes of recompressed expanded graphite have a lamellar structure formed of superposed carbon planes orthogonal with respect to direction C (and therefore parallel to direction A).

By way of variation, a cell 10 according to the invention as shown in FIG. 3 is used. Such a cell has microgrooves 15 at the interface 5 between the electrode 12 and the membrane 11, and microgrooves 14 at the interface 6 between the electrode 13 and the membrane 11. For the sake of clarity, only a few microgrooves are shown (they can be provided in a large number). These microgrooves, which typically have a diameter of the order of 10 μm, are obtained by inserting threads between the layers of graphite and vermiculite that are formed, which threads are subsequently removed after consolidation of said layers. The microgrooves are used to circulate the reagents continuously through the electrochemical cell.

The cells 1 and 10 shown are obtained by uniaxial compression of layers of graphite and vermiculite according to a direction C which is orthogonal with respect to the interfaces between said layers. It is also possible to produce cells according to the invention by uniaxial compression of layers of graphite and vermiculite according to a direction parallel to the interfaces between said layers, or alternatively by compression according to several directions, and especially by triaxial compression according to three orthogonal directions. This last version of the method according to the invention provides a cell whose electrodes are weakly anisotropic and have good electronic conductivity in all directions. This feature, which is particularly advantageous in the case of porous, that is to say weakly compressed, electrodes, improves the efficiency of the electrochemical cell because all the potential reaction sites within the electrodes are suitably supplied with electrons.

The invention relates also to a method for producing a mold, which is shown in FIGS. 4 and 5. According to the invention, there are placed in the centre of a triaxial press 23 a model 24 representing the components that are to be produced using the mold, a separating sheet 25 which surrounds the model and divides it substantially into two halves, a network 26 of tubes which are provided in the separating sheet or in the vicinity thereof and are intended to form, within the mold, channels for receiving a heating/cooling liquid, and a tube (not shown) which extends at least between the model and a plane of intersection of two columns of the press in order to form a pouring shaft inside the mold.

Each column 34, 35, 36 of the press is filled with a layer of expanded graphite 32 on either side of the model, then with a layer of expanded vermiculite 31 which covers the layer of expanded graphite 32. The material used to form the layer of expanded graphite is, for example, constituted solely of expanded natural graphite; the material used to form the layer of vermiculite is, for example, constituted solely of expanded vermiculite.

The layers so formed are subsequently compressed by displacing the six plates of the press towards the centre thereof, the plates of column 35 being actuated according to direction C, those of column 34 according to direction B and those of column 36 according to direction A, until they meet to form a cube.

The mold that has been formed is subsequently removed from the press and then opened by its joining plane 33 delimited by the separating sheet 25. There is thus obtained a mold 20 in two parts 21, 22. The sheet 25, the tubes 26, the pouring tube and the model 24 are removed from the mold. Each mold part or half comprises an inner consolidated layer 32a of recompressed expanded graphite, which delimits one half 29 of the cavity for the component to be molded, and an outer consolidated layer 31a of recompressed expanded vermiculite, which encases the layer 31a and forms an insulating protection for the mold. The quantities of expanded graphite and vermiculite introduced into the press to form the corresponding layers are chosen in dependence on the dimensions of the press and the desired final density of the consolidated layers 31a and 32a.

Each mold half 21, 22 also comprises semi-cylindrical channels 27, 28 which form, with the associated channels of the other mold half, a circuit for circulation of a liquid for heating/cooling the mold. At least one of the mold halves 21, 22 further comprises a pouring shaft 30 which extends between one face of the mold and the cavity 29 and through which the material to be molded, preferably in liquid form, can be introduced or injected.

It should be pointed out that an independent circuit for circulation of heating/cooling liquid can be formed in the layer 32a of each mold half. Such a method is preferred because it guarantees that the circuits are perfectly leak-tight. It should also be pointed out that it is possible to insert, in each layer of expanded graphite, before any compression has been carried out, resistors (cables) which are to be connected to a current generator in order to heat the mold.

In order to obtain a mold according to the invention it is also possible to use a uniaxial press as shown in FIG. 1, to form a layer of expanded graphite on either side of a model, then to form two layers of expanded vermiculite on either side of the layer of graphite, and then to compress the layers according to single direction. There is obtained a parallelepipedal mold, of which only two opposing faces are insulated by a consolidated layer of vermiculite.

By way of variation, the three layers so consolidated (provided with the model) are put back into the uniaxial press so that the layers of vermiculite extend parallel to compression direction C of the press, and then the layers are recompressed. The mold so obtained is formed of three superposed layers which have been compressed, in succession, according to two orthogonal directions. It is possible to repeat the operation in order to compress the layers according to a third direction orthogonal with respect to the first two directions. Before each of the second and third compression operations, it is possible to form two new layers of expanded vermiculite on either side of the layers previously consolidated. There is then obtained a parallelpipedal mold, four faces of which are insulated by a layer of vermiculite if two compression operations only have been carried out, or all six faces of which are insulated if three compression operations have been carried out.

In the case of a mold at least one face of which does not have insulating protection of vermiculite, the temperature within the mold can likewise be controlled and adjusted by contact of said face(s) with a body having a controlled temperature or by exposure (without contact) of said face(s) to a source of infra-red radiation (then by thermal conduction inside the consolidated layer of graphite). Accordingly, by virtue of the valuable thermal properties (conductivity, diffusivity, absorptivity, selectivity) of the recompressed expanded graphite, it is not necessary to produce or insert, within the layer of graphite, a heating/cooling circuit in order to be able to control the temperature of the mold around the cavity.

The mold 20 shown is composed of two parts 21, 22 each forming one half of the cavity for the components to be molded. However, the invention also permits the production of one-piece molds (such molds have to be destroyed in order to remove the component therefrom) or molds in three or more parts.

The invention relates also to a method for producing a heliothermal converter, shown in FIG. 6. In a uniaxial press 62 having a plate 51 of square or rectangular cross-section there are formed a layer of expanded vermiculite 52 and then a layer of expanded graphite 53 in which there is placed at least one tube 54 having the shape of a coil, rake, spiral or any other suitable shape. The tube 54 is arranged parallel to the plate 62 and at the upper face of the layer of vermiculite, during the formation of the layer of graphite. The position of said tube in the layer and the total height of the layer of graphite are such that the tube 54 remains entirely covered with graphite once the layers have been consolidated. Owing to its shape and length, the tube 54 is distributed uniformly in the layer of graphite, and its two ends each abut a wall of the press 51.

The layers of expanded vermiculite and graphite are then compressed until they are consolidated. The press used has a plate provided with an impression matrix having a plurality of ribs 69 in order to impress capture forms into the absorption face of the converter.

In this embodiment, the recompressed graphite is used, for the production of a heat exchanger, as filling material which, owing to its thermal qualities (absorptivity, diffusivity) and mechanical qualities (consolidation by simple compression), allows both the efficiency of the exchanger to be improved and the production method therefor to be simplified. The choice of compression ratio is the result of a compromise between the consolidation of the layers (the ratio must be higher than a minimum consolidation ratio), the desire for a gain in mass for the exchanger (a low compression ratio, that is to say a low density of the layer, allows a smaller quantity of expanded graphite to be provided and the final mass of the exchanger to be limited), the thermal and energy performances of the exchanger (the absorptivity and diffusivity of the consolidated layer of graphite depend on its density), the presence of the tube 54 (which it is advisable not to squash), etc. A compression ratio corresponding to a density of the consolidated layer of graphite of from 30 to 200 kg/m³ (and in all cases less than 400 kg/m³) is preferred.

It should be pointed out that it is possible to compress the layers according to several directions, but this complicates the production method unnecessarily in view of the (small) gain obtained in terms of the efficiency of the converter.

The consolidated layers (see FIG. 7) are removed from the press. The consolidated layer of graphite 56, provided with the tube 54, produces a heat exchanger in the form of a thick panel; the consolidated layer of vermiculite 55 forms an insulating base which covers the bottom face of the exchanger. The absorption face 70 of the exchanger has a plurality of straight grooves or slits such as the groove 68 of triangular cross-section, the groove 66 of semi-circular cross-section, the groove 67 of square cross-section, all having a width and depth of less than 1 cm and preferably from 1 to 5 mm. These grooves or slits form capture forms, which are impressed at the same time as the consolidation of the layers by compression and in such a manner as to be inserted between two limbs of the tube 54. It should be pointed out that the impression of the capture forms can optionally be carried out after consolidation of the layers, during a subsequent operation. The capture forms 66, 67, 68 improve both the efficiency of radiant heating/cooling and the mechanical strength of the exchanger.

Insulating plates, each constituted by a layer of recompressed expanded vermiculite, are provided and fixed (by any suitable means, such as adhesive bonding or screwing) on the four edges of the consolidated layers 55 and 56 to form an insulating frame 64.

Each end of the tube 54 opens out at the surface of an edge of the consolidated layer of graphite 56. Before the insulating frame is assembled, a bore is provided opposite said tube end, which bore passes through the plate of the insulating frame 64 adjacent to said edge. A straight tube is provided in the bore and connected to the end of the tube 54. The two ends of the tube 54 can accordingly be connected, respectively, to means for supplying cold liquid coolant and to means for recovering the liquid coolant heated by solar energy.

The insulating case produced by the base 55 and the frame 64 of vermiculite is inserted in a leak-tight casing comprising a base 59, side walls 60 and an upper edge 63 extending towards the outside of the casing. Such a casing protects the case of vermiculite—which is hydrophilic—from humidity and atmospheric influences. The casing is closed, at the top, by a plate 58 of toughened glass which is fixed to the upper edge 63 or the side walls 60 of the casing, by any suitable means. Tightness between the glass plate and the casing is ensured by means of a peripheral seal 57 of silicone, for example.

In a variant shown in FIG. 8, the channel 61 for circulation of the liquid coolant has been formed directly in the mass of the consolidated layer of graphite. To this end, a destructible tube, made of wax or polystyrene, for example, is placed in the layer of expanded graphite during its formation. After compression and consolidation of the layer of graphite, the destructible tube is destroyed. In this example, the recompressed graphite constitutes the rigid structure of the heat exchanger (and not a filling material). The imposed compression ratio is therefore chosen to be sufficiently high to confer good mechanical strength on the assembly, in dependence on the stresses to which it is subjected: pressure of the liquid coolant, hydraulic connection, large variations in temperature.

It should be pointed out that the channel portions that pass through the consolidated layer of vermiculite (which is hydrophilic) in order to connect the channel 61 to the means for supplying liquid coolant and for recovering said liquid coolant, are formed by inserted straight metal tubes, as in the preceding example. It should likewise be pointed out that the converter 65 shown does not have capture forms according to the invention, but it is advantageous to provide such capture forms.

The method for producing the converters 50 and 65 has a large number of advantages:
the absorber and the heat exchanger are integrated in one and the same structure (the layer of graphite), which is assembled in a single consolidation operation, in contrast to earlier known converters which comprise, as absorber, a metal plate which has to be produced (by extrusion, drawing, etc.), treated against corrosion and oxidation and covered with a deposit of chromic oxide (highly toxic), then welded to the exchanger. The converter 65 even comprises an absorber-exchanger produced entirely in a single operation (the tubes are omitted),
the heat exchanger and the insulating base are formed and assembled in a single operation,
the mass of the converter obtained is far lower than that of known converters,
the efficiency of the converter according to the invention is far superior to the efficiency of earlier converters, given that
the heat exchange surface corresponds to the entire circumference of the tube 54 or the channel 61,
the recompressed expanded graphite has good optical selectivity (S=2) in the range of infra-red waves which generate the greenhouse effect in the converter. This quality renders covering with black chromium unnecessary,
the recompressed expanded graphite has better thermal diffusivity (magnitude which measures the aptitude to transmit heat and is a function of the thermal conductivity, the calorific capacity and the density of the material) than the metals used in earlier techniques to produce the absorbent plate. In particular, a recompressed expanded graphite having a density of the order of 100 kg/m³ has a diffusivity that is 3 times greater than that of copper for a density 90 times lower, 2 times greater than that of silver for a density 100 times lower, and 4 times greater than that of aluminium for a density 27 times lower,
the recompressed expanded graphite has lower thermal inertia than metals, so that the response time of the converter is improved,
the recompressed expanded graphite has a coefficient of (linear) expansion in the face of thermal variations that is low, especially 8 times lower than that of copper, 11 times lower than that of aluminium, and 6 times lower than that of silver,
the efficiency of the converter 65 (without copper tube) is particularly improved owing to the direct exchange of heat between the absorber (the graphite) and the liquid coolant,
the efficiency of the converter 50 is improved by the presence of forms for capturing infra-red waves,
the cost price of the converters is low (in particular that of the converter 65 owing to the omission of the copper tubes),
graphite and vermiculite are chemically inert materials which do not give out gases after compression and are neither dangerous nor toxic, including in the form of fine flakes (whether they be particles of expanded graphite or vermiculite or fragments of recompressed graphite or vermiculite). They are more readily recyclable.

In a version of the invention (which can be applied to the two examples of converters above), electric resistors are also arranged in the layer of expanded graphite, before consolidation thereof. These resistors serve to subject the heat exchanger, punctually, to a temperature greater than or equal to 70° C. in order to prevent any risk of contamination of the exchanger with bacteria (such as legionellae). The method according to the invention therefore allows the converter to be equipped with anti-bacterial treatment means in a simple, rapid and economic manner.

It goes without saying that the invention can be the subject of numerous variations in respect of the embodiments described above and shown in the figures. In particular, the method according to the invention can be applied to the production of other composite objects.

1.-49. (canceled)

50. A method for producing a composite object comprising at least two distinct parts having different properties and/or functions, which comprises:
forming at least one layer comprising more than 70 wt. % of an expanded material selected from expanded graphites,
forming at least one other layer comprising more than 70 wt. % of another expanded material selected from expanded vermiculites,
and then compressing together the layers so formed so as to consolidate them, each consolidated layer corresponding to one of the parts of the object.

51. The method as claimed in claim 50, wherein the layers are formed to be adjacent.

52. The method as claimed in claim 50, wherein the layers formed are compressed together according to several directions.

53. The method as claimed in claim 52, wherein the layers formed are compressed together according to three orthogonal directions.

54. The method as claimed in claim 50, wherein the layers formed are compressed according to a single direction.

55. The method as claimed in claim 54, wherein the direction of compression (c) is substantially orthogonal with respect to an interfacial plane between said layers.

56. The method as claimed in claim 50, wherein the layers formed are subjected to a single compression operation according to each direction (A, B, C).

57. The method as claimed in claim 50, wherein the layers formed are subjected to a single compression operation.

58. The method as claimed in claim 50, wherein the layers formed are subjected to a plurality of distinct compression operations according to at least one direction.

59. The method as claimed in claim 58, wherein there are carried out, according to that direction, a first compression operation suitable for consolidating the layers formed in order to allow them to be handled and, subsequently, a second compression operation suitable for conferring a desired density on one of said layers.

60. The method as claimed in claim 50, wherein, during compression of the layers formed, there are impressed into at least one face, called an outer face, of at least one layer of graphite open recessed forms, called capture forms, which are suitable for trapping infra-red waves.

61. The method as claimed in claim 50, wherein there is used as expanded graphite an expanded natural graphite.

62. The method as claimed in claim 50, wherein the layer of vermiculite formed comprises less than 30 wt. % additives selected from perlite, expanded materials obtained from oxides such as SiO₂ or Al₂O₃, kandites, illites, smectites, kaolinites.

63. The method as claimed in claim 50 for producing an electrochemical cell, wherein a layer of expanded vermiculite is formed between two layers of expanded graphite, and then the layers so formed are compressed together.

64. The method as claimed in claim 63, wherein the layers formed are compressed together according to three orthogonal directions.

65. The method as claimed in claim 63, wherein the layers formed are compressed in such a manner that the two consolidated layers of graphite have a density of from 30 to 60 kg/m³.

66. The method as claimed in claim 63, wherein, for at least one of the layers of graphite, microgrooves are formed on one face of said layer, called an inner face, that is oriented towards the layer of vermiculite, by placing destructible or removable threads between the layer of expanded graphite and the layer of expanded vermiculite during their formation, said threads being destroyed or removed once the layers have been consolidated.

67. The method as claimed in claim 63, wherein heating/cooling members are incorporated into at least one of the layers of expanded graphite during its formation.

68. The method as claimed in claim 63, wherein during compression of the layers formed, there are impressed into at least one face, called an outer face, of at least one layer of graphite open recessed forms, called capture forms, which are suitable for trapping infra-red waves, and there are impressed into at least one outer face of at least one layer of graphite capture forms having at least one front dimension of from 1 μn to 5 mm and a depth of from 1 μm to 1 mm.

69. The method as claimed in claim 63, wherein each layer of graphite formed comprises less than 20 wt. % of a powder of a catalytic material, such as a catalytic metal or metal oxide.

70. The method as claimed in claim 63, wherein the layer of vermiculite formed comprises lyophilized enzymes.

71. The method as claimed in claim 50 for producing a mold, wherein a model is covered with a layer of expanded graphite, then a layer of expanded vermiculite that covers at least part of the layer of graphite is formed, and then the layers so formed are compressed together.

72. The method as claimed in claim 71 for producing a casting mold, wherein the layers formed are compressed together in such a manner that the consolidated layer of graphite has a density greater than 100 kg/m³.

73. The method as claimed in claim 71, wherein heating/cooling members are placed in the layer of expanded graphite during its formation.

74. The method as claimed in claim 71, wherein at least one channel suitable for receiving a heating/cooling fluid is formed directly in the mass of graphite by placing at least one destructible or removable tube in the layer of expanded graphite during its formation, said tube(s) being destroyed or removed once said layer has been consolidated.

75. The method as claimed in claim 71, wherein during compression of the layers formed, there are impressed into at least one face, called an outer face, of at least one layer of graphite open recessed forms, called capture forms, which are suitable for trapping infra-red waves, and the layer of vermiculite is formed in such a manner as to leave at least one face of the consolidated layer of graphite, called an outer face, visible when the mold is in use, and wherein there are impressed into at least one outer face of the layer of graphite capture forms having at least one front dimension of from 1 mm to 2 cm and a depth of from 1 mm to 10 cm.

76. The method as claimed in claim 50 for producing a heliothermal converter, wherein a layer of expanded graphite is formed, in which there is provided at least one channel suitable for receiving a liquid coolant, a layer of vermiculite is formed, which layer covers at least part of the layer of graphite and leaves uncovered at least one face thereof, called an absorption face, and then the layers so formed are compressed together.

77. The method as claimed in claim 76, wherein at least one permanent tube is placed in the layer of expanded graphite.

78. The method as claimed in claim 76, wherein at least one destructible or removable tube is placed in the layer of expanded graphite, said tube(s) being destroyed or removed once the layer of graphite has been consolidated.

79. The method as claimed in claim 76, wherein there are impressed into the absorption face of the consolidated layer of graphite capture forms having front dimensions of from 10 μm to 1 cm and a depth of from 1 mm to 1 cm.

80. A composite object comprising at least two distinct parts having different properties and/or functions, wherein one of the parts comprises a consolidated layer comprising more than 70 wt. % of a recompressed expanded material selected from expanded graphites, and wherein another of the parts comprises another consolidated layer comprising more than 70 wt. % of another recompressed expanded material selected from expanded vermiculites.

81. The object as claimed in claim 80, which is an electrochemical cell and comprises at least one consolidated layer of recompressed expanded vermiculite inserted between two consolidated layers of recompressed expanded graphite.

82. The object as claimed in claim 81, wherein the consolidated layers of graphite have a density of from 30 to 60 kg/m³.

83. The object as claimed in claim 81, wherein at least one of the consolidated layers of graphite has microgrooves on one face, called an inner face, that is oriented towards the layer of vermiculite.

84. The object as claimed in claim 81, wherein at least one of the consolidated layers of graphite incorporates heating/cooling members.

85. The object as claimed in claim 81, wherein at least one of the consolidated layers of graphite has, on at least one outer face, impressed open recessed forms, called capture forms, which are suitable for trapping infra-red waves.

86. The object as claimed in claim 85, wherein the capture forms have at least one front dimension of from 1 μm to 5 mm and a depth of from 1 μm to 1 mm.

87. The object as claimed in claim 81, wherein the consolidated layer of vermiculite comprises lyophilized enzymes.

88. The object as claimed in claim 80, which is a mold and comprises at least one consolidated layer of recompressed expanded graphite delimiting a cavity corresponding to an object to be reproduced by molding, and a consolidated layer of recompressed expanded vermiculite covering at least part of said layer of graphite.

89. The object as claimed in claim 88, which is a casting mold and wherein the consolidated layer of graphite has a density greater than 100 kg/m³.

90. The object as claimed in claim 88, wherein the consolidated layer of graphite incorporates heating/cooling members.

91. The object as claimed in claim 88, wherein the consolidated layer of graphite comprises at least one channel which has been formed directly in the mass of graphite and is suitable for receiving a heating/cooling fluid.

92. The object as claimed in claim 88, wherein the consolidated layer of graphite has at least one face, called an outer face, that is visible when the mold is in use, and wherein at least one outer face of the consolidated layer of graphite comprises impressed open recessed forms, called capture forms, which are suitable for trapping infra-red waves.

93. The object as claimed in claim 92, wherein the capture forms have at least one front dimension of from 1 mm to 2 cm and a depth of from 1 mm to 10 cm.

94. The object as claimed in claim 80, which is a heliothermal converter and comprises at least one consolidated layer of recompressed expanded graphite comprising at least one channel suitable for receiving a liquid coolant, and a consolidated layer of recompressed expanded vermiculite covering the layer of graphite with the exception of at least one face thereof, called an absorption face.

95. The object as claimed in claim 94, wherein the channel(s) has/have been formed directly in the mass of graphite.

96. The object as claimed in claim 94, wherein the channel(s) is/are constituted by tube(s) accommodated in the layer of graphite.

97. The object as claimed in claim 94, wherein the consolidated layer of graphite has, on its absorption face, impressed open recessed forms, called capture forms, which are suitable for trapping infra-red waves.

98. The object as claimed in claim 97, wherein the capture forms have at least one front dimension of from 10 μm to 1 cm and a depth of from 1 mm to 1 cm.
